(19) United States
(12) Patent Application Publication
(10) Pub. No.: US 2008/0182056 A1
(43) Pub. Date: Jul. 31, 2008

(54) Hollow Microsphere Particles

(76) Inventors: Eric Bakker, West Lafayette, IN (US); Katarzyna Wygladacz, West Lafayette, IN (US); Nan Ye, West Lafayette, IN (US); Chao Xu, West Lafayette, IN (US)

Correspondence Address: Townsend and Townsend and Crew LLP, Two Embarcadero Center, 8th Floor, San Francisco, CA, 94111, US

(21) Appl. No.: 11/669,085

(22) Filed: Jan. 30, 2007

(51) Int. Cl. G01N 33/00; B29D 22/00; B32B 27/00

(52) U.S. Cl. 428/36.8; 428/35.7; 523/223; 73/23.2; 73/53.01

(57) ABSTRACT

Disclosed herein are novel monodispersed hollow microsphere particles having a general shell-core structure with a monodispersity of from about 0.1% to about 50%, a diameter in the range of from about 3 μm to about 30 μm and a shell thickness of from about 0.1 μm to about 5 μm. The particles generally have a hydrophobic exterior shell matrix and a hydrophilic interior core, wherein the interior core may further comprise a number of materials or a cargo. Also disclosed are micro sensors comprising the hollow microsphere particles, methods for forming the sensors, as well as methods for using the sensors.

### FIELD OF THE INVENTION

The present invention, in general, relates to uniform dimensioned hollow microsphere particles. More particularly, the present invention relates to novel monodisperse, core-shell hollow microsphere particles, methods for making thereof, and methods for using thereof.

### BACKGROUND OF THE INVENTION

Nano and micro scale hollow spherical particles have attracted considerable attention in recent years. They have great potential utilities in material science and medicine. Both inorganic and polymeric hollow microspheres having a general core-shell structure have been reported in the literature. For example, Tan et al. have reported the fabrication of double-walled microspheres for the sustained release of doxorubicin (Journal of Colloid Interface Sci. 291, 135-143), and Pekarek et al. have reported double-walled polymer microspheres for controlled drug release (Nature 367, 258-260).

Among the published microspheres, hollow microsphere particles made from metal (e.g. gold), metal oxides (e.g. Al₂O₃, TiO₂, ZrO₂), silica, polymers (e.g. poly(methylmethacrylate), poly(N-isopropylacrylamide), polyorganosiloxane, poly(acrylamide)/poly(acrylic acid) (PAAM/PAAC), poly(styrene), poly(3,4-ethylenedioxythiophene) (PEDOT), polyaniline (PANI), polypyrrole (PPY) and composites (e.g. ZnS, CdS) have been fabricated with various diameters and wall thickness.

Prior art methods for generating core-shell microspheres generally involve either physiochemical or chemical processes. In the former, an organic or inorganic substance is precipitated at the core interface during solvent evaporation or adsorption by means of electrostatic or chemical interactions. In the latter, the fabrication of core-shell particles by chemical processes utilizes various multi-step polymerization reactions. The first step is to prepare seeds (templates) such as polymer beads, colloids, surfactant vesicles, emulsion droplets, or amphiphilic diblock polymers. Subsequently, a monomer is added and polymerized via emulsion, microemulsion, or suspension methods. Calcinations or solvent etching is used to remove the template materials. In most cases, however, the formation of a uniform shell surrounding the core, as well as control of the shell thickness are difficult to achieve because polymerization can not be restricted to the surface of the templates.

Although the templating method is commonly used for preparing core-shell hollow particles, capabilities of this approach is very limited because, in most cases, the material(s) that need to be encapsulated in the microspheres are not suitable templates. In fact, the majority of studies were devoted to investigating the morphology of the core-shell microspheres.

Im et al. (Nature Mater. 4, 671-675 (2005)) have reported on the preparation of macroporous capsules-polymer shells with controllable holes in their surfaces, which may be useful for incorporating chemically more labile proteins. However, after loading with functional materials, these holes must be closed by thermal annealing (95° C.) or by solvent treatment. Such conditions are often harsh for the encapsulated cargo, and may cause damage of the cargo (e.g. denaturation of proteins).

Therefore, there still exists a need for a method that can generate hollow microsphere particles with an uniform dimension under mild, chemically non-reactive conditions.

### SUMMARY OF THE INVENTION

In view of the above, it is an object of the present invention to provide novel nano or micro scale hollow microsphere particles having a core-shell structure. It is also an object of the present invention to provide a method for fabricating monodisperse nano or micro scale hollow microsphere particles under physically and chemically mild conditions.

Accordingly, in a first aspect, the present invention provides a plurality of hollow particles, wherein each individual particle comprises an hydrophilic interior core; and an exterior shell matrix comprised of a polymeric material. The plurality of hollow particles are substantially spherical in shape, have a monodispersity of from about 0.5% to about 50%, have a diameter in the range of from about 3 μm to about 30 μm and a shell thickness of from about 0.1 μm to about 5 μm. The plurality of hollow particles may be homogenous or heterogeneous.

In a second aspect, the present invention provides a micro sensor for sensing an analyte dissolved in a solution environment, comprising a hollow particle having a semi-permeable hydrophobic exterior shell; a hydrophilic interior core containing a buffer with a predetermined concentration; and a sensing element disposed in the interior core. The hollow microsphere particle has a substantially spherical shape, a size of from about 8 μm to about 15 μm, a core diameter of from about 5 μm to about 10 μm, and a shell thickness of from about 1 μm to about 3 μm.

In a third aspect, the present invention provides a method for forming a micro sensor capable of sensing the presence of a predetermined analyte in a micro environment, comprising the steps of:
1) providing a hollow particle generator for generating a hollow particle wherein the hollow particle comprises a hydrophobic polymer matrix exterior shell and a hydrophilic interior core capable of carrying a sensing element in a buffer, and wherein the particle is from about 3 μm to about 30 μm in size, the exterior shell is about 1 μm to about 5 μm in thickness, the exterior shell is from about 1 μm to about 5 μm in thickness;
2) determining an amount of buffer to be included in the hydrophilic interior core based on a reaction equilibrium between the buffer and the analyte; and
3) forming a hollow particle by the particle generating means, wherein the sensing element and the buffer are disposed in the interior core,
   whereby when the sensor encounters the analyte in the environment, the sensing element generates a signal to indicate that the analyte is detected

In a fourth aspect, the present invention provides a method for detecting a carbon dioxide in a micro-environment, comprising the steps of:
1) providing a micro sensor according to embodiments of the present invention, wherein the sensing element is capable of sensing the presence of carbon dioxide to generate a measurable signal;
2) disposing the sensor in the micro-environment; and
3) measuring the signal from the sensing element,
   wherein the signal corresponds to a concentration of the carbon dioxide in the micro-environment.

In a fifth aspect, the present invention provides a method for delivering a biologically active agent to a target, comprising
1) providing a plurality of particles according to embodiments of the first aspect of the present invention, wherein the interior core of at least one hollow particle further comprises the biologically active agent, and
2) releasing the particle to the target, wherein the active agent is released to the target in a controlled release.

Other aspects and advantages of the invention will be apparent from the following description and the appended claims.

### BRIEF DESCRIPTION OF DRAWINGS

FIG. 1 shows a schematics of an exemplary particle generator for generating hollow microsphere particles of the present invention.

FIG. 2 shows an exemplary picture of microshpere particles in a microdroplet leaving the suspension chamber of the particle generator.

FIG. 3 shows a flow cytometry single-parameter histogram that depicts the microsphere size variation of exemplary poly(urethane)-based microspheres according to the present invention.

FIG. 4a-b show Cryo-FESEM images of the fabricated core-shell hollow microspheres. a-b, Morphology of the microspheres incorporated into the etched wells of an optical fiber bundle. c-d, images of sliced core-shell particles deposited on the cryo holder. Microsphere composition: 1,1″-dioctadecyl-3,3,3′,3′-tetramethylindocarbocyanine perchlorate (DiIC18) blended with poly(styrene) blended with 5 wt % bis(2-ethylhexyl)sebacate (shell); aqueous solution of the hydrophilic dye HPTS (core).

FIG. 5 shows a Fluorescence images of randomly chosen microspheres doped with the hydrophilic dye HPTS (green, in the core) and lipophilic DiIC18 (red, in the shell) deposited on a glass support.

FIG. 6 shows 3D rendering of the fluorescence emission spectra collected from a typical microsphere excited with blue (top, HPTS emission spectrum) and green light (bottom, DiIC18 emission spectrum), respectively.

FIG. 7 shows 3D renderings of the fluorescence emission spectra of a hollow microsphere with encapsulated bovine serum albumin derivatized with fluorescein isothiocyanate.

FIG. 8 shows the response characteristic of exemplary carbon dioxide sensing core-shell microparticles according to the present invention. The particles were fabricated with the hydrophilic pH indicator HPTS and the indicated concentrations of sodium bicarbonate. Carbon dioxide diffuses across the lipophilic shell to change the pH in the particle core according to established buffer equilibria, which is measured by fluorescence. The solid lines describe theoretically predicted response behavior. Good agreement of experiment and theory suggests that the particle core contains the measured amount of sensing components.

### DETAILED DESCRIPTION

Having summarized various aspects of the present invention, reference will now be made in detail to the description of the invention as illustrated in the drawings. While the invention will be described in connection with these drawings, there is no intent to limit it to the embodiment or embodiments disclosed therein. On the contrary, the intent is to cover all alternatives, modifications and equivalents included within the spirit and scope of the invention as defined by the appended claims.

In a first aspect, a hollow microsphere particle according to the present invention generally comprises an hydrophilic interior core and an exterior shell matrix comprised of a polymeric material.

The hollow microsphere particle may have a diameter in the range of from about 3 μm to about 30 μm, preferably from about 5 μm to about 15 μm, more preferably 12 μm, and a shell thickness of from about 0.1 μm to about 5 μm, preferably from about 0.5 μm to about 2.5 μm, more preferably about 1 μm.

In one embodiment, the microsphere particle has a diameter of about 12 μm, and a shell thickness of about 1 μm.

The interior core may comprise a number of materials, including, but not limited to water, an aqueous dye, an enzyme, an antibody, an aptamer, a biologically-active cargo, a sensing element, an indicator dye, a complexing agent or any combinations thereof.

Exemplary aqueous dyes may include Fluorescein, HPTS, SNAFL, or any other aqueous dye commonly known in the art.

Exemplary biologically-active cargo may include drugs, or any other commonly known biologically-active cargos.

Exemplary enzymes may include glucose oxidase, alkaline phosphatase or horseradish peroxidase, or any other commonly known enzymes.

Exemplary antibodies may include polyclonal or monoclonal IgG, or any other antibodies commonly known in the art.

Exemplary aptamers may include RNA aptamers, DNA aptamers, peptide aptamers, or any other types of aptamers commonly known in the art.

Exemplary sensing elements may include sodium picrate.

Exemplary indicator dyes may include HPTS or SNAFL.

Exemplary complexing agents may include calcium green, Fura-2 or any water soluble complexing agent.

In the above mentioned dyes, biological materials, antibodies, enzymes, aptamers, sensing elements, or complexing agents, it is to be understood that while only currently known examples are given, the invention is not so limited and any future discovered or isolated aptamers, antibodies, enzymes, and etc. may also be included in the interior core of a microsphere particle of the present invention so long as the size of the material is compatible with a microsphere particle of the present invention.

The exterior shell matrix may comprise a number of materials, including, but not limited to polyurethane, a hydrophilic polyurethane (PU), a polystyrene (PS), poly(tetrafluoroethylene), silicone rubber, a poly(methyl methacrylate-decyl methacrylate), other polyacrylates or methacrylates with variable substituent chain lengths, plasticized polyvinylchloride, or any combinations thereof.

The exterior shell matrix may be lipophilic, hydrophilic, porous or semi-permeable.

In some embodiments, the exterior shell may also be multi-layered. In those embodiments where the exterior shell is multi-layered, the different layers may be directly in contact, forming a direct laminate, or there may be an interlayer space wherein the space may be occupied by a fluid. Such multi-layered hollow microsphere particles may be formed, for example, by a specialized particle generator with the capability of generating an additional concentric stream for each additional layer (an example of such generator is described in a patent application being concurrently filed with the present application). In the case where the layers form a direct laminate, a single concentric stream is required for each layer of the laminate. Care must be taken that adjacent layers in the concentric stream do not intermix under the particle forming conditions. This may be controlled by varying the miscibility, viscosity, composition and flow conditions of adjacent streams. An important requirement is that the process should operate at low Reynolds number where flow is laminar or nearly so. Provision of interlayers (such as aqueous interlayers between lipophilic streams) reduces concerns about adjacent layer mixing but may require a more complex particle generator apparatus providing a separate concentric stream for each interlayer as well as each other layer. Use of such an apparatus together with judiciously selected materials, permits generation of uniform particles of great structural complexity in a mass production process.

The exterior shell matrix may further comprise a dopant, including, but not limited to a lipophilic dye, a fluorescent dye, a lipophilic ion-exchanger, a suitable complexing agent, or any combinations thereof.

Exemplary lipophilic dye may include Nile Red or other lipophilic dyes commonly known in the art.

Exemplary fluorescent dye may include a proton-selective fluoroionophore such as the nile blue derivative N,N-diethyl-5-(octadecanoylimino)-5H-benzo[a]phenoxazin-9-amine (ETH 5294), 4-{[9-(dimethylamino)-5H-benzo[a]phenoxazin-5-ylidene]amino}benzeneacetic acid 11-[(1-butylpentyl)oxy]-11-oxoundecyl ester (ETH 2439) or 4-{[9-(dimethylamino)-5H-benzo[a]phenoxazin-5-ylidene]amino}benzoic acid 11-[(1-butylpentyl)oxy]-11-oxoundecyl ester (ETH 5418), or any commonly used fluorescent dye.

Exemplary lipophilic ion-exchangers may include tetraphenylborates, substituted dodecacarboranes, tetralkylammonium salts, tetraalkylphosphonium salts or any other commonly used ion-exchanger.

Exemplary complexing agents may include any of the numerous lipophilic receptors/ionophores commonly used in ion-selective electrodes and corresponding optodes that may aid in selectively transporting the analyte of interest across the particle shell.

The interior core may also comprise a dopant, or a cargo, including, but not limited to pharmaceuticals, buffers, cells, culture media, chemical feedstocks, catalysts, magnetic materials, or any combinations thereof.

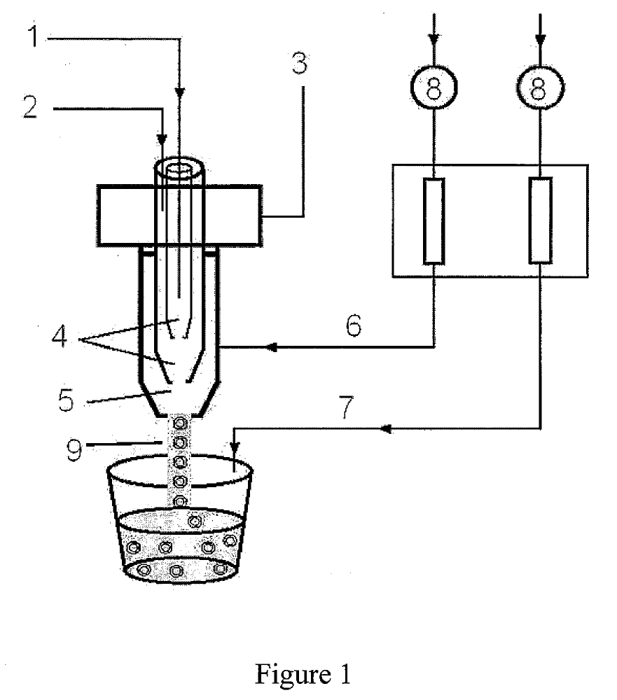

To manufacture a hollow microsphere particle of the present invention, a particle generating device such as the exemplary particle generator shown in FIG. 1 may be used (an exemplary particle generator was obtained from Beckman Coulter having features as outlined below, which is described in a patent application that is being concurrently filed with the present application). In one embodiment, an exemplary microsphere particle generating device may include two syringe pumps (not shown) for delivering a core solution 1 and a shell solution 2 through a conduit within the body of the particle generator. A pair of coaxially arranged ceramic flow nozzles 4 may be mounted on the exiting end of the particle generator conduit for shaping the exiting stream. During operation, the core solution stream 1 is directed through a first nozzle and then into a second nozzle, and the shell solution 2 is directed into the second nozzle such that it surrounds the core stream from the first nozzle entering through the space between the first nozzle and the second nozzle. As the combined streams exit the second nozzle, the shell solution stream 2 contacts the core solution stream 1 to form a sheath enveloping the core solution stream in a coaxial arrangement.

To discretize the coaxial core-shell stream, a frequency generator 3 may be mounted on the particle generator. In one embodiment, the frequency generator is a vibrator that vibrates the ceramic nozzles 4 at high frequency to break the emerging core-shell solution stream into discrete droplets, thereby “discretizing” the core-shell stream into individual core-shell microsphere particles.

A pressurized solution bottle (not shown) regulated by a pressure regulator 8 may also be connected to the particle generator for providing a carrier solution, preferably deionized water. The nascent microsphere particles are first suspended in the carrier solution inside a suspension chamber 5. The carrier solution then forms a sheath around the nascent microsphere particles for carrying the particles in a continuous flow from the suspension chamber 5 into a collection vial placed below the nozzles. In this way, the nascent microsphere particles are carried from the suspension chamber to the collection vial in a continuous flow of protective aqueous carrier stream 9 without being exposed to air.

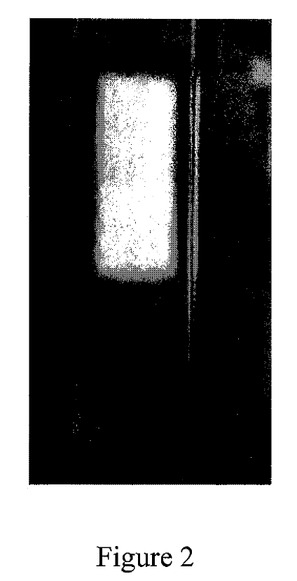

FIG. 2 shows a high speed photographic image of a stream of nascent microsphere particles leaving the suspension chamber of a microsphere particle generator in an aqueous sheath. The image is captured by placing a strobed light emitting diode (LED) next to the stream. It can be clearly seen from the image that discretized particles are evenly spaced in a line within the carrier aqueous sheath stream.

To prevent the nascent microspheres from aggregating, soap 7 may be added to the collection vial.

The discrete hollow microsphere particles generated are uniform in size and have a monodispersity of from about 0.1% to about 50%, preferably about 5%, and more preferably less than 2%.

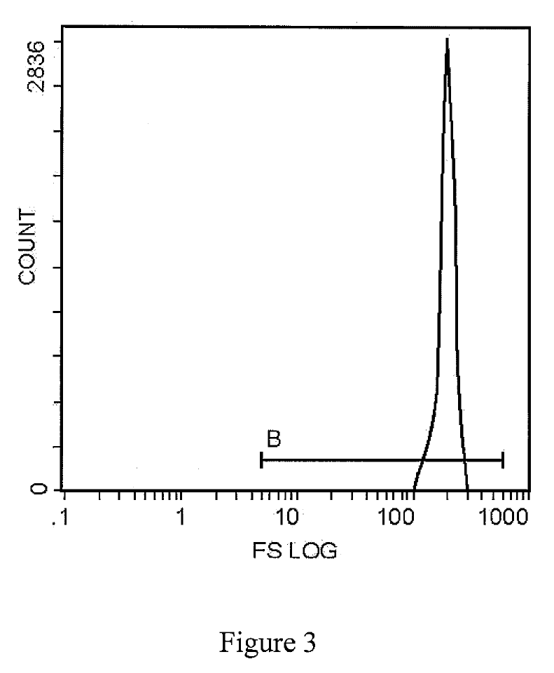

FIG. 3 shows a histogram of a size distribution of hollow microsphere particles according to embodiments of the present invention. The size of the particles were measured by flow cytometry. It can be seen that hollow particles of the present invention have very small variation in size.

The hollow microsphere particles are believed to have many utilities in material science and medicine. However, most utilities involving microparticles remain speculative due to the difficulties in their production. The inventors of the present invention have conceived and reduced to practice a novel type of chemical sensors utilizing the hollow microsphere particles according to embodiments of the present invention.

Accordingly, in a second aspect, the present invention provides a micro sensor for sensing an analyte dissolved in a solution environment, comprising: 1) a hollow microsphere particle having a hydrophilic interior core containing a buffer with a predetermined concentration; 2) a hydrophobic semi-permeable shell; and 3) a sensing element disposed in the interior core, wherein the particle has a substantially spherical shape, a size of from about 8 μm to about 15 μm, a core diameter of from about 7.9 μm to about 14.9 μm, more preferably from about 5 μm to about 10 μm, and a shell thickness of from about 0.1 μm to about 3 82 m.

In some embodiments, the sensing element is capable of sensing a biological analyte. In other embodiments, the sensing element is capable of sensing a chemical analyte.

In one embodiment, the micro sensor is capable of sensing a carbon dioxide level in a micro-environment, wherein the sensing element is HPTS and the interior core of the hollow microsphere particle further comprises a predetermined amount of sodium carbonate buffer.

In another embodiment, the micro sensor is capable of sensing a level of creatinine in a solution, wherein the sensing element is sodium picrate at elevated (alkaline) pH in the core of the particle. Creatinine forms a highly colored adduct with picrate under these conditions which is known as the colorimetric Jaffe reaction. The Jaffe reaction does not give a fluorescence signal change. An additional fluorescent dye placed either in the core or the shell of the particle and whose excitation spectrum overlaps with the absorbance spectrum of that of the Jaffe reaction may be used to give fluorescence signals. This is known as the inner filter effect. The hollow particle shell needs to be permeable to creatinine, which may be accomplished by doping the shell with a lipophilic hydrogen bond forming receptor.

In a third aspect, the present invention also provides a method for forming a micro sensor capable of sensing the presence of a predetermined analyte in a micro environment. The method comprising the general steps of: 1). providing a hollow particle generator for generating a hollow particle according to the first aspect of the present invention; 2) determining an amount of buffer to be included in the hydrophilic interior core based on a reaction equilibrium between the buffer and the analyte; and 3) forming a hollow particle by the particle generating means, wherein the sensing element and the buffer are disposed in the interior core, whereby when the sensor encounters the analyte in the environment, the sensing element generates a signal to indicate that the analyte is detected.

In a fourth aspect, the present invention also provides a method for detecting a carbon dioxide in a micro-environment, comprising the general steps of 1) providing a micro sensor according to an embodiment of the second aspect of the present invention; 2) disposing the sensor in the micro-environment; and 3) measuring a fluorescence intensity of the sensing element, wherein the fluorescence intensity corresponds to a concentration of the carbon dioxide in the micro-environment

In a fifth aspect, the present invention further provides a method for delivering a biologically active agent to a target, comprising the general steps of 1) providing a plurality of particles according to embodiments of the first aspect of the present invention, wherein the interior core of at least one hollow particle further comprises the biologically active agent, and 2). releasing the particles to the target, wherein the active agent is released to the target in a controlled release.

Among the methods of controlled release of particle contents are photochemically initiated decomposition reactions that change the permeability of the shell membrane to the core components. For example, the shell polymer may incorporate photocleavable moieties such as a 2-nitrobenzyl group in the polymer backbone. Exposure of the hollow particle to near-TV light cleaves the polymer at the photocleavable group. This reduction in the structural integrity of the shell may of itself increase shell permeability, or the shell may be designed as a block copolymer (such as a block copolymer of polystyrene and poly(n-butyl methacrylate) where the blocks are joined by a photocleavable group. Photocleavage permits the resultant sub-polymers to redistribute into micro-phase separated regions, increasing the shell permeability. Similar effects are possible where the link is thermally labile and the controlling element is a temperature change.

Other methods of controlled release rely on the presence of photolabile compounds within the core of the particles. For example, if the core were to contain a caged proton, such as 2-hydroxyphenyl 1-(2-nitrophenyl)ethyl phosphate or 1-(2-nitrophenyl)ethyl sulfate, exposure to light would change the pH in the particle core, exposing the shell polymers to protonation at groups with appropriate pKa. The change in ionization of the polymer components would then alter the cohesive forces among the polymer strands, modifying shell permeability.

In one embodiment, the target is a patient, the biologically active agent is a drug, and the step of releasing the particles to the target further comprises administering the particles to the patient.

### EXAMPLES

To further illustrate the various aspects and embodiments, the following specific examples are provided.

### Materials and Methods

### 1. Materials

PVC, PU and DOS were purchased from Fluka (Milwaukee, USA). DiIC18, HPTS and FITC were from Molecular Probes, (Eugene, Oreg.). PS (Acros Organic, New Jersey, USA), methylene chloride (Fisher, Fair Lawn, N.J.), cyclohexanone (99.8%) (Sigma-Aldrich, St. Louis, Mo.), hemocyanin (MP Biomedicals, Inc, Solon, Ohio), bovine serum albumin (Sigma-Aldrich, St. Louis, Mo.) were reagent grade purchased from the indicated suppliers. Copolymer methyl methacrylate-dodecyl methacrylate poly(MMADMA) was synthesized in our lab according to the procedure published elsewhere (Qin et al. Plasticizer-free polymer membrane ion-selective electrodes containing a methacrylic copolymer matrix. Electroanal. 14, 13751381 (2002), the relevant portions of which are incorporated herein by reference).

### 2. Conjugation of FTTC With Hemocyanin and Bovine Serum Albumin

The preparation of FITC-hemocyanin and FITC-BSA was based on the method described elsewhere (Voss et al. Detection of protease activity using a fluorescence-enhancment globular. BioTechniques 20, 286-291 (1996), the relevant portions of which are incorporated herein by reference). Briefly, protein (hemocyanin or BSA, 10 mg/mL) was dissolved in water with an equal weight of K2CO3 to adjust the pH to 10.5. FITC was added (2 mg/mL) and reacted at 37° C. with mild stirring for 24 h in an amber bottle. The derivatized product was purified using a PD-10 column (Amersham Biosciences, Uppsala, Sweden). The resulting product was analyzed for the degree of substitution.

### 3. Core-Shell Hollow Microsphere Particle Preparation

Fluorescent hollow microspheres were generated using a custom built sonic particle casting device. The ceramic tips had diameter orifices of 36 μm and 78 μm, and the flow rates for the core and shell solution were both kept at 1 mL/min with a water flow at 0.75 mL/min. The piezoelectric crystal was operated at 10 kHz. Microspheres suspended in the receiving water phase were collected in 20 mL glass vials. The particles were cured for 2 d in water before characterization.

Typically a total mass of 90 mg hydrophobic shell compounds including the polymeric matrix and, optionally, plasticizer and 0.015 mmol/kg DiIC18 was dissolved in 2.5 mL cyclohexanone and diluted with 50 mL of methylene chloride. Either 2 mg/mL HPTS dissolved in water; fluorescein isothiocyanate (FITC) conjugated with hemocyanin or BSA in TRIS buffer pH 7.8; or 2 mg/mL HPTS in 0.04 (0.005) M NaHCO₃ (for carbonate sensors) served as the aqueous core solution.

### 4. Instrumentation

Fabricated microspheres were characterized by: fluorescent microscopy (Nikon Eclipse E400 microscope equipped with two CCD cameras EDC 1000L (Electrim. Corp., Princeton, N.J.) in combination with a PARISS Imaging Spectrometer (Light Form, Belle Mead, N.J., Nikon E800 microscope with an infinity fluorescence imaging SPOT RTslider digital camera (Diagnostic Instruments, Inc.) with 40×magnification; Slow cytometry (Beckman Coulter EPICS XL flow cytometer); and Cryo Held Emission Scanning Electron Microscopy. For cryo-FESEM sample droplets were deposited on the etched distal face of the optical fiber bundle or were directly dried down on carbon tape on the cryo sample holder. The sample was prepared for cryo imaging using a Gatan Alto 2500 cryo system. The holder with the fiber or directly with the adhered particles was plunged into liquid nitrogen and a vacuum pulled prior to transfer to the cryoprechamber. It was sputter-coated with Pt for 120 s. Samples were imaged with an FEI NOVA nanoSEM FESEM at 3 kV.

### 5. Size Distribution and Measurements

Microspheres size was established using cryo-FESEM images as well as based on the recorded fluorescence spectra according to the method reported in Tsagkatakis et al. and Wygladacz et al. (Tsagkatakis et al., Monodisperse plasticized poly(vinyl chloride) fluorescent microspheres for selective ionophore-based sensing and extraction. Anal. Chem. 73, 6083-6087 (2001), and Wygladacz et al., Imaging fiber microarray fluorescent ion sensors based on bulk optode microspheres. Anal. Chim. Acta 532, 61-69 (2005), the relevant portions of which are incorporated herein by reference).

### Example 1

### Production of Monodisperse Hollow Microsphere Particles

A custom built microsphere particle generator was used to generate hollow microspheres whose interior compartments can be controllably doped with known amounts of hydrophilic reagents. The particle generator is schematically shown in FIG. 1, the components and operation of which are described above in the Methods for manufacturing hollow microsphere particles section.

Briefly, the particle generator consists of two syringe pumps for delivering core and shell solutions, a pressurized solution bottle for the aqueous sheath flow, a flow chamber, a pressure regulation unit, a frequency generator, and a metal flow chamber. The individual solution streams from the syringe pumps are directed to two coaxial flow nozzles in the metal flow chamber and surrounded by the aqueous sheath flow. This results in three concentric solution streams, with the organic solvent containing non-crosslinked hydrophobic polymer acting as the intermediate stream that separates the aqueous interior and exterior (sheath) flows. A periodic destabilization of this solution stream by a constant frequency oscillation driven by a piezoelectric crystal placed above the suspension chamber leads to the formation of uniform microdroplets within the continuous sheath stream. These droplets eventually form polymeric hollow particles upon loss of organic solvent during a curing step in aqueous solution in the presence of a surfactant (PEG) to avoid agglomeration. The flow rates of the three streams and the frequency of the piezoelectric crystal are adjustable and hollow particles can be cast with controllable size and shell thickness. The casting conditions are visibly monitored using a stereomicroscope and a strobed light emitting diode. A typical hollow microdroplet stream recorded during casting is presented in FIG. 2.

### Example 2

### Characterization of Hollow Microsphere Particles

Core-shell microspheres fabricated from either PU or PS as the shell material exhibited a spherical shape and a sufficiently high HPTS fluorescence intensity. The size distribution of the PU core-shell microsphere particles was evaluated by flow cytometry. The sharp peak on the flow cytometry histogram shown in FIG. 3 indicates a high monodispersity of the core-shell microspheres fabricated here.

The microsphere morphology was characterized by cryo-FESEM since classical SEM gave unreliable images, likely because of melting problems caused by the electron beam. For the purpose of this experiment, microspheres were deposited on the etched wells of an optical fiber bundle.

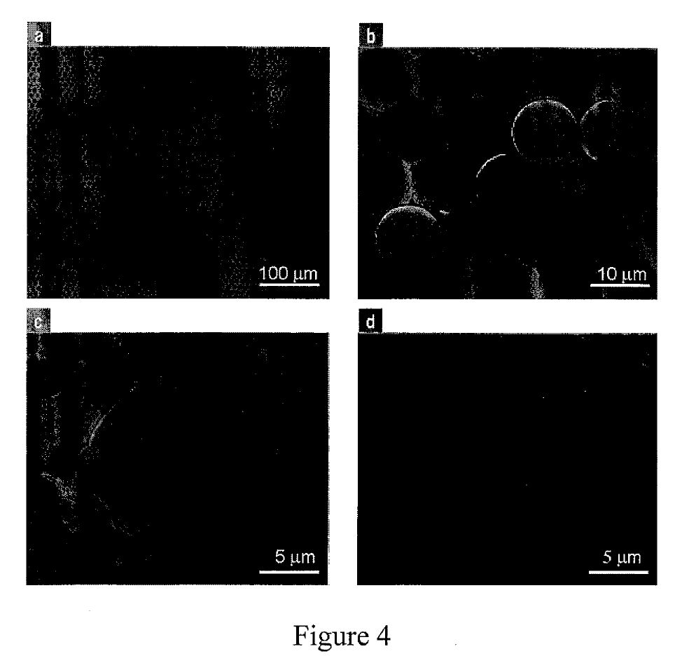

A scanning electron micrograph of the PS-based core-shell microspheres is presented in FIG. 4a and b. Note that the microspheres are smooth, spherical and uniform in size. The established microsphere size of 12 μm is in good agreement with the data obtained by fluorescent microscopy (see below).

To determine the microsphere shell diameter the PS-based microspheres were deposited on the cryo holder, sliced, and imaged by cryo-FESEM (FIGS. 4c and d). They were found to contain a large void in the center of the particles, as expected. The shell was noticeably thin (about 1 μm), in accordance with fluorescence microscopy data (see below). The observed particle deformations may be caused by the pressure on the thin walls of the microspheres during the slicing or drying/cooling process.

Two fluorescent dyes were used to demonstrate the presence of the core-shell structure by their spatially resolved spectral signatures in fluorescence microspectroscopy. The hydrophilic pH indicator HPTS was doped into the particle core, while the lipophilic dye DiIC18 was incorporated into the shell material during casting. Blue light excited both dyes with emission peaks at 517 and 540 nm, respectively, while green light gave only a fluorescence signal from DiIC18 at 612 nm.

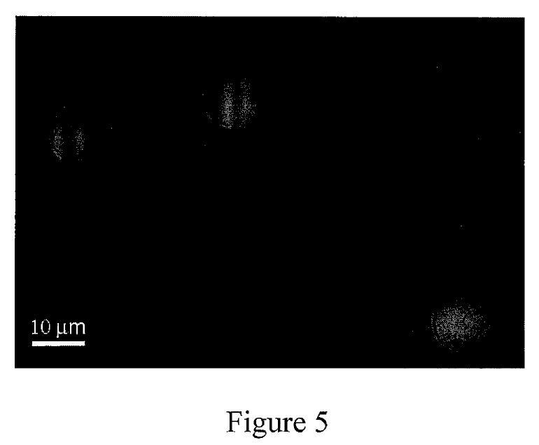

Typical fluorescence images of the PS-based microspheres are presented in FIG. 5. Note that both the core and shell of the microspheres exhibit the expected spherical shape. Bright green color in the image corresponds to HPTS in the core while red color indicates the reference dye DiIC18 located in the shell. Note that the particle cores are perfectly centered and are surrounded by a very thin and uniform polymeric shell (ca. 1 μm shell thickness for a 12 μm particle as estimated by fluorescence microscopy). This implies a uniform doping of the particles with both dyes. Time studies revealed that the particle structure was maintained for at least three weeks after casting. Hollow microspheres made of PU exhibited similar characteristics (data not shown), suggesting that both materials are useful for the stated purpose.

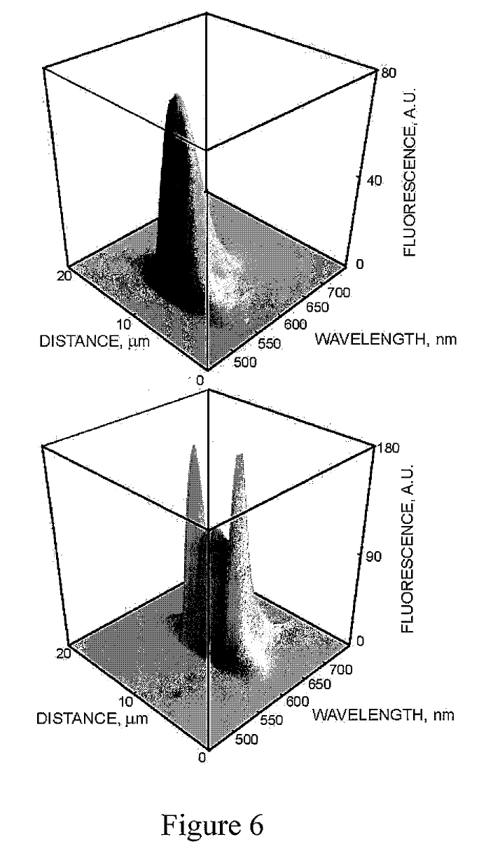

FIG. 6 illustrates a 3D rendering of the fluorescence emission spectra recorded from a representative core-shell particle based on PU and containing the two dyes mentioned above. Microspheres excited with blue light exhibit a strong emission peak at 517 nm attributed to HTPS (FIG. 6 top). Note that this peak has a regular particle emission peak shape, which means that the hydrophilic dye is only concentrated in the core of the microsphere. Green light only excites the lipophilic DiIC18. Under these conditions an unusually shaped spectral image (see FIG. 6 bottom) with a maximum intensity at 612 nm was recorded. This confirms that the reference dye is concentrated in the shell only.

The relationship between the fluorescence intensity and the particle core diameter was also established to assess the quantitative loading of the dye in the particle core. Particles with core diameters ranging from 5 to 20 μm were fabricated and studied for this purpose (data not shown). In agreement with expectations, a linear relationship between the core size and recorded intensity was observed, independent of the material used for shell preparation (PS or PU).

### Example 3

### Hollow Microsphere Particles Containing Biological Material

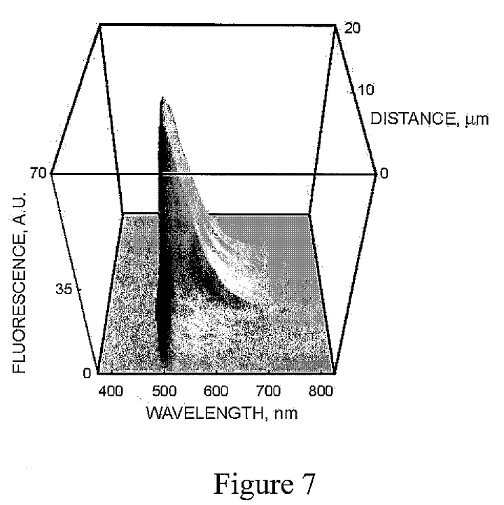

Fluorescent proteins were incorporated into the microspheres core as model biological compounds. Fluorescein isothiocyanate (FTIC) linked to hemocyanin and bovine serum albumin (BSA) with a spectral signature at 525 nm were chosen as the core dopant with PS as the shell material. FIG. 7 displays the 3D renderings of the fluorescence emission spectra collected from a hollow microspheres doped with BSA-FTIC. Note that the cast microspheres exhibited a fluorescence characteristic similar to the isolated compounds, suggesting that biological components can be successfully incorporated into such hollow particles. Optical characteristic of the core-shell particles containing FTIC linked to hemocyanin was analogical to those containing BSA-FTIC (data not shown). The lack of relatively harsh chemical reaction conditions or temperatures in the procedure introduced here makes it attractive for the encapsulation of relatively fragile compounds relevant in biochemistry and biosensing.

### Example 4

### Carbon Dioxide Micro Sensor

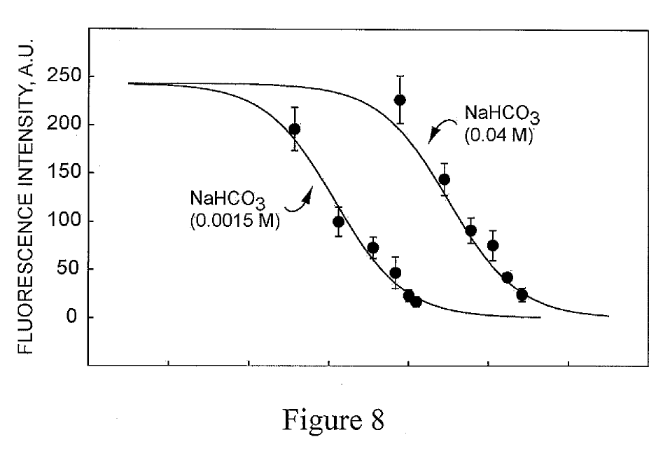

The hydrophilic dye HPTS explored in the above example is a pH indicator, and can be utilized for carbon dioxide sensing if the dye solution also contains a calculated concentration of sodium bicarbonate and is separated from the sample solution by a semi-permeable membrane. Carbon dioxide can diffuse across the membrane and change the pH of the indicator dye solution by the established buffer equilibrium between the diffusing acid and bicarbonate. This principle was explored as an early model for chemical sensing using the hollow microspheres established here. Two sets of particles were explored, each containing the same concentration of HPTS but different concentrations of sodium bicarbonate. FIG. 8 shows the corresponding fluorescence responses as a function of the carbon dioxide concentration in the surrounding solution, together with the two theoretically expected curves calculated on the basis of established buffer equilibria. The excellent correspondence between theory and experiment again suggests that the composition of the particle core can be accurately controlled during the fabrication process and maintained during measurement in contact with aqueous samples.

1. A plurality of hollow microsphere particles, wherein:
each individual particle comprises:
a hydrophilic interior core; and
a exterior shell matrix comprised of at least one layer of a polymeric material, wherein the plurality of hollow particles are substantially spherical in shape, have a monodispersity of from about 0.1% to about 50%, a diameter in the range of from about 3 μm to about 30 μm and a shell thickness of from about 0.1 μm to about 5 μm.

2. The plurality of hollow microsphere particles of claim 1, wherein:
the exterior shell matrix of at least one hollow particle is lipophilic.

3. The plurality of hollow microsphere particles of claim 1, wherein:
the exterior shell matrix of at least one hollow particle is semi-permeable.

4. The plurality of hollow microsphere particles of claim 1, wherein:
the exterior shell matrix of at least one hollow microsphere particle is comprised of a polyurethane, a hydrophilic polyurethane, a polystyrene, poly(tetrafluoroethylene), silicone rubber, a poly(methyl methacrylate-decyl methacrylate), plasticized polyvinylchloride, or combinations thereof.

5. The plurality of hollow microsphere particles of claim 4, wherein
the exterior shell matrix of the at least one hollow microsphere particle further comprises a dopant selected from the group consisting of a lipophilic dye, a fluorescent dye, a lipophilic ion-exchanger, a suitable complexing agent, or combinations thereof.

6. The plurality of hollow microsphere particles of claim 5, wherein the dopant is a tetraphenylborate derivative cation-exchanger.

7. The plurality of hollow microsphere particles of claim 5, wherein the complexing agent is a hydrogen bond forming receptor for transporting the analyte of interest.

8. The plurality of hollow microsphere particles of claim 1, wherein:
the interior core of at least one hollow microsphere particle comprises an aqueous solvent.

9. The plurality of hollow microsphere particles of claim 1, wherein:
the interior core of at least one hollow microsphere particle further comprises an aqueous dye, a biological material, an enzyme, an antibody, an aptamer, a sensing element, an indicator dye, a complexing agent or combinations thereof.

10. The plurality of hollow microsphere particles of claim 9, wherein:
the biological material further comprises one selected from glucose oxidase, horseradish peroxidase, alkaline phosphatase, or combinations thereof.

11. The plurality of hollow microsphere particles of claim 9, wherein:
the complexing agent or the indicator dye is alkaline picrate.

12. The plurality of hollow microsphere particles of claim 1, wherein:
the interior core of at least one hollow microsphere particle further comprises a biologically active agent, and wherein the particle is capable of controlled release, whereby the active agent is released into an environment over a predetermined period of time.

13. The plurality of hollow microsphere particles of claim 1, wherein:
the shell thickness is about 1 μm.

14. The plurality of hollow microsphere particles of claim 1, wherein the diameter is about 12 μm.

15. The plurality of hollow microsphere particles of claim 1, wherein the hollow microsphere particles are homogeneous.

16. The plurality of hollow particles of claim 1, wherein the hollow microsphere particles are heterogeneous.

17. A micro sensor for sensing an analyte dissolved in a solution environment, comprising:
a hollow microsphere particle having a semi-permeable hydrophobic exterior shell;
a hydrophilic interior core containing a buffer with a predetermined concentration; and
a sensing element disposed in the interior core,
wherein the particle has a substantially spherical shape, a size of from about 8 μm to about 15 μm, a core diameter of from about 5 μm to about 10 μm, and a shell thickness of from about 1 μm to about 3 μm.

18. The micro sensor of claim 17, wherein the sensing element is capable of sensing a biological analyte.

19. The micro sensor of claim 17, wherein the sensing element is capable of sensing a chemical analyte.

20. The micro sensor of claim 17, wherein the sensing element is a pH indicator and the core comprises a pH buffer.

21. The micro sensor of claim 20, wherein the sensing element is HPTS and the interior core further comprises a predetermined amount of sodium bicarbonate.

22. The micro sensor of claim 21, wherein the exterior shell is comprised of a polyurethane, a hydrophilic polyurethane, a polystyrene, poly(tetrafluoroethylene), silicone rubber, a poly(methyl methacrylate-decyl methacrylate), or plasticized polyvinylchloride.

23. A method for forming a micro sensor capable of sensing the presence of a predetermined analyte in a micro environment, comprising:
providing hollow microsphere particle generator for generating a hollow microsphere particle wherein the hollow microsphere particle comprises a hydrophobic polymer matrix exterior shell and a hydrophilic interior core capable of carrier a sensing element in a buffer, and wherein the particle is from about 3 μm to about 30 μm in size, the exterior shell is from about 1 μm to about 5 μm in thickness;
determining an amount of buffer to be included in the hydrophilic interior core based on a reaction equilibrium between the buffer and the analyte; and
forming a hollow microsphere particle by the particle generating means, wherein the sensing element and the buffer are disposed in the interior core,
whereby when the sensor encounters the analyte in the environment, the sensing element generates a signal to indicate that the analyte is detected.

24. The method of claim 23, wherein the analyte is carbon dioxide.

25. The method of claim 24, wherein the sensing element is HPTS and the buffer is sodium bicarbonate buffer.

26. The method of claim 23, wherein the signal has an intensity corresponding to a concentration of the analyte in the environment in accordance with the predetermined equilibrium between the buffer and the analyte.

27. The method of claim 23, wherein the exterior shell is comprised of a polyurethane, a hydrophilic polyurethane, a polystyrene, poly(tetrafluoroethylene), silicone rubber, a poly(methyl methacrylate-decyl methacrylate), plasticized polyvinylchloride, or combinations thereof.

28. A method for detecting a carbon dioxide in a micro-environment, comprising:
providing a micro sensor according to claim 21;
disposing the sensor in the micro-environment; and
measuring a fluorescence intensity of the sensing element,
wherein the fluorescence intensity corresponds to a concentration of the carbon dioxide in the micro-environment.

29. A method for delivering a biologically active agent to a target, comprising:
providing a plurality of particles according to claim 12; and
releasing the particles to the target, wherein the active agent is released to the target in a controlled release.

30. The method of claim 29, wherein the target is a patient and the biologically active agent is a drug, and wherein the delivering step comprises administering the particles to the patient.
